(19) United States
(12) Patent Application Publication
(10) Pub. No.: US 2020/0170847 A1
(43) Pub. Date: Jun. 4, 2020

(54) PRINTABLE COATING ON A SUBSTRATE

(71) Applicant: Kimberly-Clark Worldwide, Inc., Neenah, WI (US)

(72) Inventors: Devin C. McElrath, Broomfield, CO (US); Michael L. Lohoff, Oshkosh, WI (US); Andrew E. Neubauer, Fremont, WI (US); Linda M. Ahles, Neenah, WI (US); Tim G. Dollevoet, Kimberly, WI (US)

(21) Appl. No.: 16/629,534

(22) PCT Filed: Mar. 29, 2019
(86) PCT No.: PCT/US19/25044

§ 371 Date: Jan. 8, 2020

Related U.S. Application Data
(60) Provisional application No. 62/650,547, filed on Mar. 30, 2018.

(51) Int. Cl. A61F 13/15; A61F 13/84; B41M 5/52

(52) U.S. Cl. CPC A61F 13/15731; A61F 13/496; B41M 5/52; A61F 13/84

(57) ABSTRACT

The present disclosure relates to a printed absorbent article and a process for making a printed absorbent article. Specifically, the printing onto the outer cover of an absorbent article may be accomplished by using a hydrophobic substrate and applying aqueous ink containing pigments to an ink receptive coating that has a thickness of about 0.1 millimeters or less and a base weight of at least 2 gsm wherein the coating absorbs liquid from the ink at a contact angle at or about zero degrees at which time the ink is printed onto the hydrophobic substrate at a printing speed of at least 500 feet per minute.

### BACKGROUND OF THE DISCLOSURE

Polymers are used extensively to make a variety of products which include blown and cast films, extruded sheets, injection molded articles, foams, blow molded articles, extruded pipe, monofilaments, fibers, and nonwoven fabrics. Many polymers that are used to form these products, such as polyolefins, are naturally hydrophobic or apolar and are chemically inert. For many uses, hydrophobicity is a disadvantage, particularly when printing with aqueous-based inks having a relatively higher surface tension than the surface energy of the polymeric substrate. For example, aqueous-based inks can have a surface tension of greater than or equal to above a surface tension energy of 45 dynes/cm, while the polymeric substrate may have a surface tension energy of about 30 dynes/cm. Surface tension is the force that holds a fluid together. Surface tension directly impacts whether a coating will wet and spread over, or retract (bead up) from a substrate. While substrate hydrophobicity may not be an issue with lower surface tension inks or solvent-based inks, still the apolar nature of the polymeric substrate will not promote good adhesion of these inks, either aqueous or solvent based, to the polymeric substrates, resulting in printed graphics that will easily rub off when exposed to shear.

Typically, the polymers used to form these products are poorly polar resulting in them being non-conducive to adhere most common ink compositions applied to the surface of the polymeric substrate. Also, these polymers are typically non-absorbent and unable to form a mechanically strong network with the ink composition after it is applied to the polymeric substrate.

Hydrophobic polymers, including polyolefins, such as polyethylene and polypropylene, may be used to manufacture polymeric fabrics which are employed in the construction of packaging articles and disposable absorbent articles such as diapers, feminine care products, incontinence products, training pants, wipes, and so forth. Such polymeric fabrics are often nonwoven fabrics prepared by, for example, processes such as melt blowing, carding, co-forming, spunbonding, and combinations thereof.

Absorbent articles, especially personal care absorbent articles, such as pantiliners, sanitary napkins, interlabial devices, adult incontinent devices, bandages, wipes, diapers, training pants, and swimming pants, typically include an outer cover made from a nonwoven polymeric fabric or alternatively the outer cover is made by printing on a plastic film, then laying a nonwoven on top of the printed plastic film. The outer cover of diapers, training pants, and swimming pants, for example, are difficult to print in a fast and economic manner that is amiable to efficient machine production.

Accordingly, there is a need to improve adhesion of inks to the outer cover of the aforementioned absorbent articles by increasing printing speeds. The current disclosure addresses this need by utilizing a process in which designs and/or graphics may be printed directly onto a small porous size or non-porous nonwoven substrate or alternatively, the designs/graphics are printed onto a plastic film and thereafter a nonwoven is placed on top of the printed film. The novel process disclosed herein may achieve fast printing speeds of at least 500 feet per minute and more without the use of a drying step thus reducing manufacturing process time and cost.

### SUMMARY OF THE DISCLOSURE

The present disclosure relates to a printed absorbent article and a process for making a printed absorbent article. Specifically, the printing onto a hydrophobic substrate may be accomplished by applying aqueous ink containing pigments to an ink receptive coating that has a thickness of about 0.1 millimeters (mm) or less and more preferably with a thickness of about 0.0015 mm with a base weight from about 2 gsm to about 4 gsm and from about 2 gsm to about 3 gsm wherein the coating absorbs liquid from the ink at a contact angle at or about zero degrees at which time the ink is printed onto the absorbent article at a printing speed of at least 500 feet per minute.

In an embodiment of the present disclosure, a printed absorbent article comprises a hydrophobic substrate; an ink receptive coating wherein the coating has a thickness of about 0.1 millimeter or less and a base weight from about 2 gsm to about 18 gsm; and applying aqueous ink containing pigments wherein the coating absorbs liquid from the ink at a contact angle at or about zero degrees at which the ink is printed onto the substrate through the coating at a printing speed of at least about 500 feet per minute.

In another embodiment of the present disclosure, a printing process for making a printed absorbent article comprises the steps: apply an ink receptive coating to a substrate wherein the coating has a thickness of about 0.1 millimeter or less and a base weight from about 2 gsm to about 18 gsm and applying aqueous ink containing pigments, whereby the coating absorbs liquid from the ink at a contact angle at or about zero degrees, to the substrate through the ink receptive coating at a printing speed of at least about 500 feet per minute

### BRIEF DESCRIPTION OF DRAWINGS

The disclosure may be more completely understood in consideration of the following detailed description of various embodiments in connection with the accompanying drawing, in which:

FIGS. 1A-B show a comparison between two disposable absorbent articles wherein the left side absorbent article shows where a wet ink has been transferred to the backside of the printed surface when wound in a roll without the presence of an ink receptive coating. And wherein the right side absorbent article shows a disposable absorbent article with the absence of any ink transferring to the backside due to the presence of an ink receptive coating.

FIGS. 2A-B show a comparison between two disposable absorbent articles wherein the left side absorbent article shows where a wet ink has been transferred to the backside of the printed surface when wound in a roll without the presence of an ink receptive coating. And wherein the right side absorbent article shows a disposable absorbent article with the absence of any ink transferring to the backside due to the presence of an ink receptive coating.

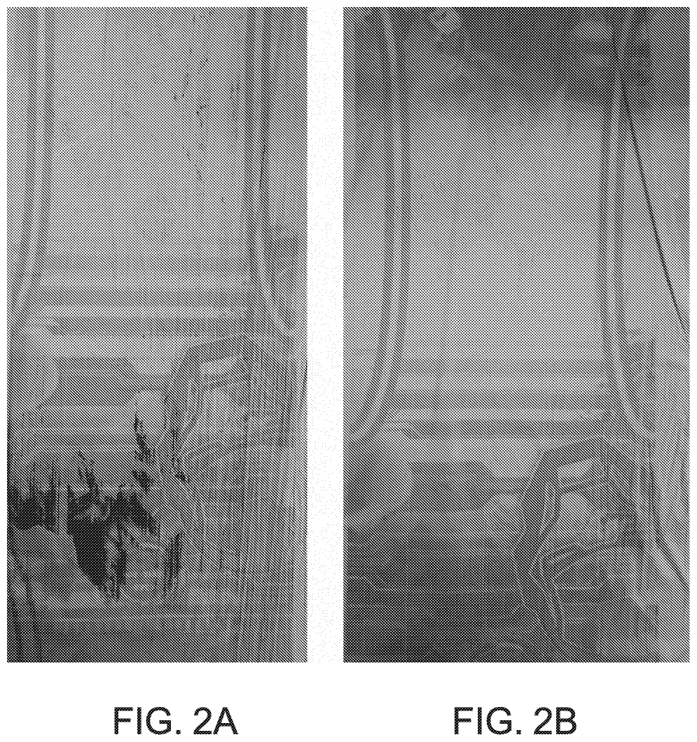

### DETAILED DESCRIPTION OF THE DISCLOSURE

When introducing elements of the present disclosure or the preferred embodiment(s) thereof, the articles “a”, “an”, and “the” are intended to mean that there are one or more of the elements.

The terms “comprising”, “including” and “having” are intended to be inclusive and mean that there may be additional elements other than the listed elements.

The term “absorbent article” refers to devices that absorb and contain body exudates, and, more specifically, refers to devices that are placed against or in proximity to the body of the wearer to absorb and contain the various exudates discharged from the body. Absorbent articles may include diapers, training pants, adult incontinence undergarments, feminine hygiene products, breast pads, care mats, bibs, wound dressing products, and the like. As used herein, the term “body exudates” includes, but is not limited to, urine, blood, vaginal discharges, breast milk, sweat and fecal matter.

The term “nonwoven” is a manufactured sheet, web or batt of directionally or randomly orientated fibers, bonded by friction, and/or cohesion and/or adhesion, excluding paper and products which are woven, knitted, tufted, stitch-bonded incorporating binding yarns or filaments, or felted by wet-milling, whether or not additionally needled. Nonwovens may include hydroentangled nonwovens. The fibers may be of natural or man-made origin and may be staple or continuous filaments or be formed in situ. Commercially available fibers have diameters ranging from less than about 0.001 mm to more than about 0.2 mm and they come in several different forms: short fibers (known as staple, or chopped), continuous single fibers (filaments or monofilaments), untwisted bundles of continuous filaments (tow), and twisted bundles of continuous filaments (yarn). Nonwoven fabrics can be formed by many processes such as meltblowing, spunbonding, solvent spinning, electrospinning, and carding. The basis weight of nonwoven fabrics is usually expressed in grams per square meter (gsm).

The term “substrate” includes any material that the inks of the present invention can be printed on. Thus, substrates of the present invention include, but are not limited to, nonwovens, films, fibrous polyolefin webs, polyolefin webs, cellulosic webs, elastomeric webs, laminates of one or more of the above or any combination of one or more of the above.

### Absorbent Article

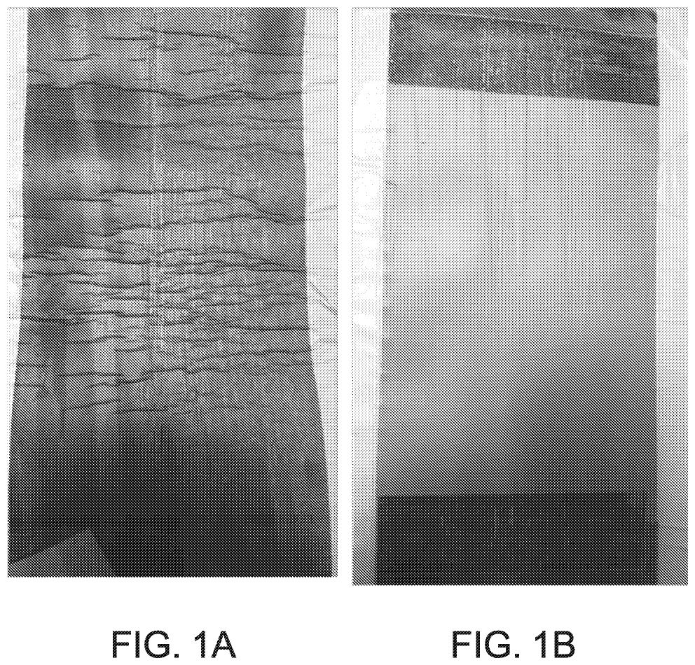

With reference to FIGS. 1A-B and 2A-B, an absorbent article is disclosed wherein the absorbent article comprises an absorbent chassis. The absorbent chassis defines a front waist region, a back waist region, a crotch region interconnecting the front and back waist regions, an inner surface which is configured to contact the wearer, and an outer surface opposite the inner surface which is configured to contact the wearer's clothing. The printing disclosed herein occurs on the outer surface of the absorbent article wherein a hydrophobic substrate is applied directly onto the outer surface. The absorbent article described in U.S. Pat. No. 6,849,067 is incorporated herein.

The outer surface (or cover) desirably comprises a material that is substantially liquid impermeable, and can be elastic, stretchable or nonstretchable. The outer surface may be a single layer of liquid impermeable material, but desirably comprises a multi-layered laminate structure in which at least one of the layers is liquid impermeable. For instance, the outer surface may include a liquid permeable outer layer and a liquid impermeable inner layer that are suitably joined together by a laminate adhesive (not shown). Suitable laminate adhesives, which may be applied continuously or intermittently as beads, a spray, parallel swirls, or the like, can be obtained from Findley Adhesives, Inc., of Wauwatosa, Wis., U.S.A., or from National Starch and Chemical Company, Bridgewater, N.J., U.S.A. The liquid permeable outer layer can be any suitable material and desirably one that provides a generally cloth-like texture. One example of such a material is a 20 gsm (grams per square meter) spunbond polypropylene nonwoven web. While it is not a necessity for outer layer to be liquid permeable, it is desired that it provides a relatively cloth-like texture to the wearer.

The inner layer of the outer surface may be both liquid and vapor impermeable, or may be liquid impermeable and vapor permeable. The inner layer is desirably manufactured from a thin plastic film, although other flexible liquid impermeable materials may also be used. The inner layer, or the liquid impermeable outer surface when a single layer, prevents waste material from wetting articles, such as bedsheets and clothing, as well as the wearer and caregiver. A suitable liquid impermeable film for use as a liquid impermeable inner layer, or a single layer liquid impermeable outer surface, is a 1.0 mil polyethylene film commercially available from Edison Plastics Company of South Plainfield, N.J., U.S.A. If the outer surface is a single layer of material, it can be embossed and/or matte finished to provide a more cloth-like appearance. As earlier mentioned, the liquid impermeable material can permit vapors to escape from the interior of the disposable absorbent article, while still preventing liquids from passing through the outer surface. A suitable “breathable” material is composed of a microporous polymer film or a nonwoven fabric that has been coated or otherwise treated to impart a desired level of liquid impermeability. A suitable microporous film is a PMP-1 film material commercially available from Mitsui Toatsu Chemicals, Inc., Tokyo, Japan, or an XKO-8044 polyolefin film commercially available from 3M Company, Minneapolis, Minn., U.S.A.

In accordance with the present disclosure, it has been discovered that the quality of a printed image on an absorbent article may be retained at printing speeds of about 500 feet per minute or more and may reach speeds up to about 2000 feet per minute or more without a separate drying step. By achieving such a fast printing speed without a drying step will reduce manufacturing build out costs and increase efficiency for the user.

Industry standards pertaining to aqueous ink technologies require that pigments absorb onto a substrate while water is removed by evaporation in drying ovens. To adequately dry these aqueous inks at current process speeds, the dry ovens would have to be at least 40 feet or more. Most manufacturing lines do not have enough space to accommodate these types of large drying ovens nor is it economically feasible. And, moreover, even if these manufacturing lines could be modified to accommodate these large drying ovens, the cost of their operation and the potential impact on print quality would be risky and too costly to justify the expense needed to implement such a manufacturing line. Therefore, a solution is needed that does not require these large drying ovens.

Surprisingly and unexpectedly, the current disclosure has found a solution to the aforementioned. The solution for absorbing the pigment to a substrate is not to evaporate a liquid with large drying ovens, but to spread and absorb the liquid, such as water and pigment(s), with a thin absorbent ink receptive coating onto the substrate.

### Ink Receptive Coating

The ink receptive coating needs to be thick enough to absorb the liquid, such as water, contained in the aqueous ink. The optimal thickness to achieve adequate removal of a liquid by the coating is about 0.1 millimeter or less.

Suitable ink receptive coatings include but not limited to are absorbent materials like talc, cellulose fibers, superabsorbent for example to aid in absorption. The coating may also contain a wetting agent like a surfactant to ensure a near zero contact angle when the inks come in contact with the coating. The coating may include other and the coating to the substrate to improve both dry and wet crockfastness.

Base weight of the ink receptive coating disclosed herein is reduced on average from about 25 to about 35% from its original weight after a liquid is absorbed. The aqueous ink containing pigments wherein the coating absorbs the liquid, such as water, from the ink is at a contact angle at or about zero degrees at which the ink is printed onto the substrate at a printing speed of at least about 500 feet per minute. It is important to note that the ink receptive coating must be a certain thickness, preferably about 0.0015 mm and have a certain base weight preferably 3 gsm so as to increase absorbency rate and wetting rate of the coating. Absorbency and wetting rates of the ink receptive coating are important measures of the current disclosure. Accordingly, the speed at which the liquid is removed from the ink on the ink receptive coating maybe 10 seconds or less. Or more preferably 5 seconds or less. Or even more preferably 2 seconds or less. Or most preferably 1 second or less. More specifically, during a manufacturing process, the liquefied pigmented ink is applied to the coating whereby the liquid, such as water, is absorbed by the coating and the ink may then be absorbed onto the substrate thus eliminating the use of a drying apparatus.

### Aqueous Ink Composition

Embodiments of the present invention includes an ink composition comprising a water-based ink. The ink composition may include a water-based polymer, a binder component, resolubility agent, pigments and optionally wax and/or lubricants. Examples of water-based inks useful in the present disclosure are available from Environmental Inks and Coatings Corporation, Morganton, N.C., under the following code numbers: EH034677 (yellow); EH057960 (magenta); EH028676 (cyan); EH092391 (black); EH034676 (orange); and EH064447 (green) and any similar inks thereof. Other suitable coatings are inkjet coatings such as NuCoat Digitall 9191 MIJ or Lubrizol DP338.

The aqueous ink composition may include polymers such as acrylics, acrylic latex, styrenated acrylics, ethylene vinyl acetate, ethylene vinyl chlorides and styrene butadiene rubbers (SBR's) or any combinations thereof.

The aqueous ink composition may include a binder component. The binder component may be from about 20 to about 45 weight percent of the ink composition. In one embodiment of the present invention, the binder component may be a polyurethane dispersion (also known herein as “PUD”). The PUD may be a high elongation, high tensile strength, high hardness, water-based polymeric dispersion.

The aqueous ink may include a wax component. The wax component may be from about 8 to about 18 weight percent of the aqueous ink composition. A wax or wax blend may be useful in the present invention. Appropriate waxes/blends include polyethylene, carnauba, paraffin, silicone oil, polypropylene, polyolefin blends and combinations thereof.

The aqueous ink may include a resolubility agent. The resolubility agent may be from about 5.0 to about 16.0 weight percent of the ink composition. Resolubility agents useful in the present invention include acrylics solutions and dispersions with a high to medium degree of carboxyl functionality. In one embodiment, medium acid number, acrylic colloidal dispersion resolubility agents are useful in the present disclosure.

The aqueous ink composition may include additional waxes and lubricants for detackification and lowering CoF. The additional wax/lubricant blend may be comprised of carnauba (the wax) and silicone oil (the lubricant). In one embodiment, the wax/lubricant blend is from about 1 to about 4 weight percent of the composition. Waxes useful in the present invention include polyethylene, polypropylenes, high density polyethylene, low density polyethylene and paraffin.

### Pigmented Aqueous Ink Compositions and Printing Techniques

The aqueous ink may include pigments. Examples of suitable pigments include, but are not limited to, Blue 15:3, Violet 23, Violet 27, Yellow 14, Yellow 74, Yellow, 83, Yellow 97, Yellow 13, Green 7, Red 2, Red 22, Red 48:1, Red 57:1, Red 122, Red 184, Red 238, Red 269, Red 49:1, Red 81:1 Red 49:2, Red 166, Red 170, Orange 5, Orange 16, Orange 46, White 7, Black 7, iron oxides, and combinations thereof. In one embodiment, from about 10 to about 16 weight percent pigments are employed, but this may vary according to the specific color and desired density. In one embodiment, pigments in a colloidal dispersion, collectively a colorant, are useful in the present disclosure.

The printing of substrates, such as woven and nonwoven fabrics and films, is well known. The printing of fabrics with inks and dyes is a common and widely used method for imparting patterns and colors to a basic fabric. Many current products, such as diapers and training pants, include printed graphics to improve their appearance. A problem with such printed products is that the printed graphics may be smeared or even removed during the handling of products during manufacturing, packaging, and use.

Pigmented inks are beneficial for use on substrates because they tend to be more resistant to leaching and mechanical rubbing than dye-based inks and thus tend to be more resistant to being removed from the surface of substrates. Pigment-based inks also have better optical density per unit weight (better “mileage”) than dye-based inks, meaning that less pigment-based ink is required to create the intensity of color. However, adhesive components are necessary to use along with pigmented inks to prevent removal of the pigment from the surface by mechanical abrasion or chemical leaching.

The industrial importance of pigment-based inks has increased in recent times. This is driven, in part, by the development of many new synthetic substrates that are incapable of being printed with conventional solvent-based or water-based inks, and consumer preferences that their goods be printed with brand identifiers, aesthetically pleasing designs or functional markings. In order to adapt pigment-based inks for use in a variety of applications, namely low surface tension substrates, others have employed high loads of volatile organic compounds (“VOC's”), thereby reducing the static and dynamic surface tension of the inks. However, volatile organic compounds such as alcohols, esters, ketones, aromatics and aliphatics create environmental hazards in their production, disposal and use. They are also expensive. One example of an ink used on a low surface tension substrate is set forth in U.S. Pat. No. 5,458,590 to Schleinz et al., which employs a solvent blend to impart the desired surface tension to the ink is incorporated herein by reference.

The aqueous ink composition may include surfactants. Surfactants may be present in the range of from about 1.0 to about 10.0 weight percent of the ink composition. Surfactants useful in the present invention include dioctyl sulfosuccinates, phosphate esters, alkoxylated alcohols, ethoxylated diols, and mixtures or blends thereof.

The aqueous ink composition may be applied to the substrate using ink jet printers, flexographic printing presses, gravure printing presses, or a combination thereof. The aqueous ink composition may be printed on different parts of an absorbent article component including, but not limited to the front waist region, a back waist region, a crotch region interconnecting the front and back waist regions, and an outer surface which may extend from the side panel to the cuffs or flaps of the absorbent article.

As mentioned previously, substrates disclosed herein are preferably very hydrophobic such as untreated polyethylene and polypropylene. Substrates may also be treated with other ingredients to improve the adherence of the coating to the substrate. Corona treating the substrate to increase the adherence of the coating to the substrate is used herein as well to improve the adherence of the coating to the substrate. Corona treatment must be done at an optimal time. If corona treatment is done too long it will destroy the properties of the coating and surface of the substrate. Other suitable printing processes may be used such as digital ink jet printing for the disclosure herein.

In one embodiment, the aqueous ink composition is applied to the substrate by flexographic or rotogravure printing. In another embodiment of the present disclosure, a design or graphic being printed onto the absorbent article is done by sparse printing on an absorbent article of a printed package. Sparse printing is defined as a low base weight of ink wherein porosity is small enough to limit the spreading of ink and/or liquid on an ink receptive coating such as a plastic film or tiny or non-porous non-woven. By printing on such a surface will provide a smoother image/graphic and/or design on the final printed absorbent article.

Other printing embodiments of the present disclosure pertain to using a metering roller or doctor blade system. A metering roller is a pitted metal roller used to transfer a metered film of ink from the ink fountain to the surface of the flexographic printing plate. A doctor blade system is a mechanical device used to remove excess ink from a printing cylinder. While it removes some ink, it allows some to stay on the cylinder, thus metering the amount of ink that leaves the cylinder.

### EXAMPLE

One example of preparing an ink absorbent article as disclosed herein is to add an absorbent layer onto a polyolefin film. One way to achieve this is by taping talc powder, tissue (cellulose fibers), and absorbent fabrics onto a polyolefin film. For comparison, the industry standard is to print on corona or plasma treated polyolefin films.

The utilization NuCoat Digitall 9191 as a print receptive coating is used to increase the print speed without the use of a dryer. Specifically it was found that using NuCoat Digitall 9191 at a base weight of 12 gsm would yield about 1400 fpm printing speed. This result was gathered without the use of a corona treater.

### Embodiments

1. A printed absorbent article comprising:
   hydrophobic substrate;
   ink receptive coating wherein the coating has a thickness of about 0.1 millimeter or less and a base weight from about 2 gsm to about 18 gsm; and applying
   aqueous ink containing pigments wherein the coating absorbs liquid from the ink at a contact angle at or about zero degrees at which the ink is printed onto the substrate through the coating at a printing speed of at least about 500 feet per minute.
2. The printed absorbent article according to claim 1, wherein the coating has a thickness of about 0.0015 millimeters and a base weight of about 3 gsm.
3. The printed absorbent article according to claim 1, wherein the substrate is a polyolefin or a plastic film.
4. The printed absorbent article of claim 1, wherein the substrate comprises paper, wood, woven fabric, textile, plastic, glass, metal, foil, or a combination thereof.
5. The printed absorbent article according to claim 1, wherein the ink receptive coating comprises talc, cellulose fibers, superabsorbent amorphous silica or a combination thereof.
6. The printed absorbent article according to claim 1, wherein the ink receptive coating comprises a surfactant.
7. The printed absorbent article according to claim 1, wherein the printing speed is about 1400 feet per minute and up to about 2000 feet per minute.
8. The printed absorbent article according to claim 1, wherein the amount of aqueous ink applied to the substrate through the coating is dependent on the amount of ink required to prepare a graphic, design or number onto the substrate.
9. The printed absorbent article according to claim 1, wherein the amount of aqueous ink absorbed by the coating after applying the aqueous ink onto the substrate is the same.
10. The printed absorbent article according to claim 1, wherein the printed absorbent article is packaged for commercial sale.
11. A printing process for making a printed absorbent article comprising the steps:
   apply an ink receptive coating to a hydrophobic substrate wherein the coating has a thickness of about 0.1 millimeter or less and a base weight from about 2 gsm to about 18 gsm and
   applying aqueous ink containing pigments, whereby the coating absorbs liquid from the ink at a contact angle at or about zero degrees, to the substrate through the ink receptive coating at a printing speed of at least about 500 feet per minute.
12. The printing process for making a printed absorbent article according to claim 11, wherein the coating has a thickness of about 0.0015 millimeters and a base weight of about 3 gsm.
13. The printing process for making a printed absorbent article according to claim 11, wherein the substrate is a polyolefin or a plastic film.
14. The printing process for making a printed absorbent article according to claim 11, wherein the substrate comprises paper, wood, woven fabric, textile, plastic, glass, metal, foil, or a combination thereof.
15. The printing process for making a printed absorbent article according to claim 11, wherein the ink receptive coating comprises talc, cellulose fibers, superabsorbent amorphous silica or a combination thereof.
16. The printing process for making a printed absorbent article according to claim 11, wherein the ink receptive coating comprises a surfactant.
17. The printing process for making a printed absorbent article according to claim 11, wherein the printing speed may be about 1000, about 1400, or about 2000 feet per minute.
18. The printing process for making a printed absorbent article according to claim 11, wherein the amount of aqueous ink applied to the substrate through the coating is dependent on the amount of ink required to prepare a graphic, design or number onto the substrate.
19. The printing process for making a printed absorbent article according to claim 11, wherein the amount of aqueous ink absorbed by the coating after applying the aqueous ink onto the substrate is the same.
20. The printing process for making a printed absorbent article according to claim 11, wherein the printed absorbent article is packaged for commercial sale.

What is claimed is:

1. A printed absorbent article comprising:
hydrophobic substrate;
ink receptive coating wherein the coating has a thickness of about 0.1 millimeter or less and a base weight from about 2 gsm to about 18 gsm; and applying
aqueous ink containing pigments wherein the coating absorbs liquid from the ink at a contact angle at or about zero degrees at which the ink is printed onto the substrate through the coating at a printing speed of at least about 500 feet per minute.

2. The printed absorbent article according to claim 1, wherein the coating has a thickness of about 0.0015 millimeters and a base weight of about 3 gsm.

3. The printed absorbent article according to claim 1, wherein the substrate is a polyolefin or a plastic film.

4. The printed absorbent article of claim 1, wherein the substrate comprises paper, wood, woven fabric, textile, plastic, glass, metal, foil, or a combination thereof.

5. The printed absorbent article according to claim 1, wherein the ink receptive coating comprises talc, cellulose fibers, superabsorbent amorphous silica or a combination thereof.

6. The printed absorbent article according to claim 1, wherein the ink receptive coating comprises a surfactant.

7. The printed absorbent article according to claim 1, wherein the printing speed is about 1400 feet per minute and up to about 2000 feet per minute.

8. The printed absorbent article according to claim 1, wherein the amount of aqueous ink applied to the substrate through the coating is dependent on the amount of ink required to prepare a graphic, design or number onto the substrate.

9. The printed absorbent article according to claim 1, wherein the amount of aqueous ink absorbed by the coating after applying the aqueous ink onto the substrate is the same.

10. The printed absorbent article according to claim 1, wherein the printed absorbent article is packaged for commercial sale.

11. A printing process for making a printed absorbent article comprising the steps:
applying an ink receptive coating to a substrate wherein the coating has a thickness of about 0.1 millimeter or less and a base weight from about 2 gsm to about 18 gsm and
applying aqueous ink containing pigments, whereby the coating absorbs liquid from the ink at a contact angle at or about zero degrees, to a hydrophobic substrate through the ink receptive coating at a printing speed of at least about 500 feet per minute.

12. The printing process for making a printed absorbent article according to claim 11, wherein the coating has a thickness of about 0.0015 millimeters and a base weight of about 3 gsm.

13. The printing process for making a printed absorbent article according to claim 11, wherein the substrate is a polyolefin or a plastic film.

14. The printing process for making a printed absorbent article according to claim 11, wherein the substrate comprises paper, wood, woven fabric, textile, plastic, glass, metal, foil, or a combination thereof.

15. The printing process for making a printed absorbent article according to claim 11, wherein the ink receptive coating comprises talc, cellulose fibers, superabsorbent amorphous silica or a combination thereof.

16. The printing process for making a printed absorbent article according to claim 11, wherein the ink receptive coating comprises a surfactant.

17. The printing process for making a printed absorbent article according to claim 11, wherein the printing speed may be about 1000, about 1400, or about 2000 feet per minute.

18. The printing process for making a printed absorbent article according to claim 11, wherein the amount of aqueous ink applied to the substrate through the coating is dependent on the amount of ink required to prepare a graphic, design or number onto the substrate.

19. The printing process for making a printed absorbent article according to claim 11, wherein the amount of aqueous ink absorbed by the coating after applying the aqueous ink onto the substrate is the same.

20. The printing process for making a printed absorbent article according to claim 11, wherein the printed absorbent article is packaged for commercial sale.
